(19) United States
(12) Patent Application Publication
(10) Pub. No.: US 2009/0060928 A1
(43) Pub. Date: Mar. 5, 2009

(54) Topical Toll Like Receptor Ligands as Vaccine Adjuvants

(76) Inventor: Jean-Claude Bystryn, New York, NY (US)

Correspondence Address: COOPER & DUNHAM, LLP, 30 Rockefeller Plaza, 20th Floor, NEW YORK, NY, 10112, US

(21) Appl. No.: 11/754,121

(22) Filed: May 25, 2007

Related U.S. Application Data
(60) Provisional application No. 60/808,786, filed on May 26, 2006.

(51) Int. Cl. A61K 39/00; A61P 35/00

(52) U.S. Cl. 424/184.1

(57) ABSTRACT

The present invention provides a method for increasing immunological response to a vaccine, comprising administering the vaccine subcutaneously to a patient in need thereof; anti administering a topical composition containing an amount of a toll like receptor ligand effective to increase immune response of the patient to the vaccine.

### CROSS REFERENCE TO RELATED APPLICATION

This application claims the benefit of U.S. Provisional Application No. 60/808,786, filed on May 26, 2006. The entire contents of the Foregoing provisional application are incorporated herein by reference.

### FIELD OF THE INVENTION

This invention relates to adjuvants for vaccines, and more particular to adjuvants for vaccines that can be applied topically, either at the site of administering the vaccine or at a site remote thereafter.

### BACKGROUND OF THE INVENTION

A critical element in constructing more effective vaccines against cancer, infections and other diseases is the availability of potent adjuvants that can boost vaccine-induced immune responses and which are safe and simple to use. Adjuvants that promote Th1 responses are particularly desirable, as these responses play a major role in protective immunity. A wide variety of adjuvants are currently available, but all have limitations. Aluminum hydroxide (alum), the only adjuvant approved for use in humans, lacks potency. The strongest adjuvant, Freund's, cannot be used in humans because it causes severe local toxicity. Dendritic cells (DC) are another potent adjuvant, but their application is limited by the cost and time required to make a custom preparation of dendritic cells for each patient. Most other adjuvants are typically admixed with the vaccine, so that each application of the adjuvant with a different vaccine must go through the difficult and lengthy FDA approval process as a new formulation. SUMMARY OF THE INVENTION

A potent adjuvant that can be used with vaccines without the need to create a new formulation would have highly desirable value. The present invention provides a method for the administration of toll-like receptor (TLR) ligands separately from the vaccine to achieve this goal. The ligand can be administered topically to skin at a site distal from the vaccine administration, to skin over the site of vaccine immunization, or possibly to a patient orally or by injection.

### BRIEF DESCRIPTION OF THE DRAWINGS

FIG. 1 shows the effect of topical imiquimod on antibody response to OVA immunization. Fig. A: Groups of mice were immunized to OVA alone or followed by topical application of imiquimod to the site of immunization 3×/wk. or to OVA in ILK-2 liposomes. One week following 4 weekly immunizations, sera was collected and tested for anti-OVA IgG antibodies by ELISA. Anti-OVA IgG responses was greater in mice treated with imiquimod than in those immunized to OVA alone. It was as strong as that induced by IL-2 liposomes as the adjuvant. Fig. B: One week following 4 weekly immunizations, IgG1, IgG2a, IgG2b, and IgG3 anti-OVA antibodies was measured in these groups by ELISA, using subclass specific antibodies as probes. IgG2a and IgG2b antibody responses were markedly boosted by imiquimod, whereas IgG1 responses were reduced compared to OVA only immunized mice. No response was seen in PBS immunized controls.

FIG. 2 shows the effect of topical imiquimod on vaccine-induced T-cell responses:. FIG. 1A: Mice were immunized to OVA with or without topical application of imiquimod treatment 1× or 3×/wk following each immunization. Spleen cells were collected from each group 1 week following 4 immunizations, and tested by ELISPOT (based on gamma-interferon release) for T-cell response to OVA positive target cells (E.G7-OVA). FIG. 2B Mice were immunized to OVA with topical application of imiquimod 3×/week or with OVA encapsulated into IL-2 liposomes. Imiquimod boosted T-cell responses more strongly than IL-2 liposomes.

FIG. 3A shows the effect of topical imiquimod on antibody (A) and cellular (B) responses to OVA immunization. FIG. 3A: Groups of mice were immunized to OVA alone or followed by topical application of imiquimod to the site of immunization 1×/wk. or to a site distal to the immunization. One week following 4 weekly immunizations, sera was collected and tested for anti-OVA IgG subclass antibodies by ELISA. Anti-OVA IgG responses was greater in mice treated with imiquimod both locally and distally than in those immunized to OVA alone. FIG. 3B: Triplicate mice were sacrificed 1 week after 4 immunizations and cellular immune responses to OVA-expressing target E.G7-OVA cells determined by ELIspot assay

### DETAILED DESCRIPTION OF THE INVENTION

Toll-like receptor (TLR) ligands as vaccine adjuvants: TLR ligands are emerging as a new class of vaccine adjuvants. TLR are expressed on a variety of antigen presenting cells (APC), and when activated stimulate the differentiation and maturation of several populations of immune cells and release of a broad range of cytokines and other immunomodulatory molecules (1-3). There are several types of TLR which differ in the type of APC on which they are expressed, on the cytokines they induce once stimulated, and consequently on the effect they have on immune responses. For example TLR7 is expressed to various degrees on all subpopulations of human APCs including myeloid and plasmacytoid DC (4,5). By contrast, TLR9 is expressed only on plasmacytoid DC and B cells (5-7).

Several ligands have been identified that can bind to and activate each of these TLRs. Each ligand activates distinct TLRs. The best studied are the imidazoquinoline molecules imiquimod and resiquimod which activate TLR7 and TLR8 (5,8,9) and certain CpG oligonucleotides (CpG ODN) which react only with TLR9 (6,7,10,1 1).

TLR 7 and 8 ligands such as imiquimod and resiquimod bind to TLR 7 and 8and by so doing activate plasmacytoid DC (pDC) and NK cells in blood (12), induce the maturation of pDC (12), rapidly upregulate the expression of costimulatory molecules CD40, CD80, and CD86 and MHC class 11, and stimulate the production of other cytokines (IL-1, IL-IR, IL-6, IL-8 and IL-12) (13). Topical application of both agents to skin attracts CD4+ CD3- pDC (14), induces the synthesis of IFN-alpha and gamma (15), and enhances maturation of Langerhans cells and their ability to present antigen (2) at the site of topical application. Imiquimod and resiquimod differ in their potency and in some of their specific immunomodulatory effects (2).

TLR 9 ligands such as CpG ODN stimulate TLR9 and cause a signaling cascade that culminates in the maturation, differentiation and proliferation of T-cells, monocytes/macrophages and natural killer cells (16). These cells secrete a number of pro-inflammatory cytokines including IL-1, IL-6, IL-12, IL-18 and interferon-gamma (16). Activation of immune cells by all of these ligands results in Th-1 dominant immune responses.

There is increasing evidence that TLRs ligands are effective vaccine adjuvants when given systemically admixed with the vaccine. The one best studied is CpG ODN. It boosts by up to three logs antibody responses to proteins such as ovalbumin, hepatitis surface antigens, and tetanus toxoid in mice (17-19). It boosts cytotoxic T lymphocyte responses and protective immunity induced by several infectious disease vaccines (malaria, hepatitis, leishmania) in primates (21-23). In a study of 19 different adjuvants in mice, CPG ODN was the one that most effectively enhanced Th-1 responses induced by a tumor-specific peptide (24).

TLR7 and TLR9 ligands also appear to be effective vaccine adjuvants. Imiquimod and resiquimod both augment specific CD4+and CD8+ T-cell responses against CMV and HIV-1 in vitro. Given systemically, both ligands enhance the strength and longevity of antigen specific CD4+ and CD8+ T-cell responses against OVA, and anti-viral protective immunity (25) in mice (11). Intramuscular resiquimod given in combination with an HIV DNA vaccine enhanced antigen-specific T-cell proliferative responses by seven-fold and Th-1 antibody responses by five-fold (13). As with CpG ODN, the immune responses are biased towards Th1 responses.

Different patterns and levels of TLRs are expressed by different types of APC such as monocytes/macrophages, myeloid and plasmacytoid DC and B cells. As a consequence, different types of immune response may be enhanced differently depending on the TLR ligand which is used and the TLR(s) which it stimulates (6, 27, 28). Thus, combinations of TLRs may induce stronger and/or broader immune responses than a single agent which is one of the applications of this invention.

TLR ligand-vaccine combinations have typically been studied with both agents given together by a variety of systemic routes including subcutaneous, intramuscular, and intranasal (16). The effect of topical application of the TLR ligand at a site distal to the vaccine, as this invention proposes, has not yet been studied to our knowledge. There are some recent reports, including our own, of TLR ligands being effective vaccine adjuvants when applied to skin directly over the site of vaccine immunization. I do not know if patents have been filed on that application.

In the first of two major discoveries that provide the foundation of this invention, we found that a TLR ligand can retain its adjuvant activity when applied topically separately from, but over the site, of vaccine immunization. We found that 5% imiquimod cream applied topically to skin over the site of a subcutaneous immunization to ovalbumin (OVA) markedly enhanced humoral and cellular immune responses against this antigen. It also markedly increased vaccine-induced tumor protective immunity against challenge with a lethal dose of OVA positive tumor cells. Importantly, the potency of imiquimod was as great as that of the strongest adjuvant we have studied to date in mice but with less local toxicity.

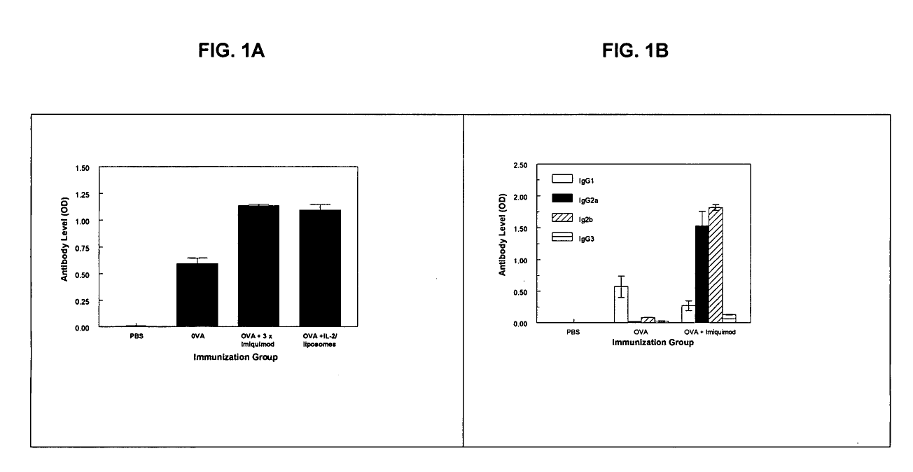

In more detail, groups of 10 C57BI/6 mice were immunized sc weekly×4 to 0.1 mg of ovalbumin (OVA). Some groups were in addition treated with 5% imiquimod cream applied topically to the immunization site once or 3×/week. Control groups were immunized to PBS, to OVA encapsulated into IL-2 liposomes (which we have previously shown is one of the strongest adjvuanrts available), or to irradiated E.G7-OVA cells (a powerful stimulator of anti-OVA T-cell responses). Serum was collected from each mouse at baseline and following the 4^{th} immunization, pooled by immunization group, and assayed for antibodies to OVA by ELISA. As shown in FIG. 1A, the overall IgG anti-OVA antibody response was considerably greater in mice immunized to OVA+topical imiquimod than in mice immunized to OVA alone. The strength of the response was as great as that obtained using IL-2 liposomes as the adjuvant but with less local toxicity (no toxicity at all with imiquimod vs. local granulomas with IL-2 liposomes).

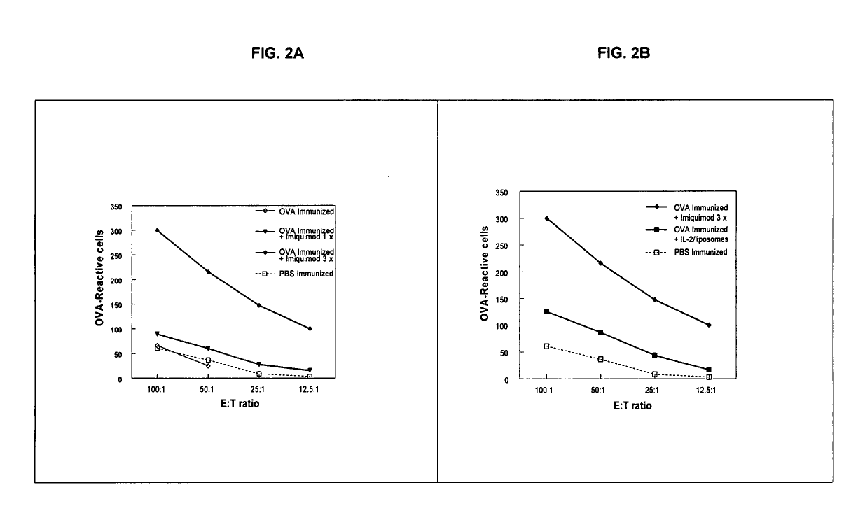

Imiquimod boosted in particular Ig2a and IgG2b responses which were almost undetectable in mice immunized to OVA alone (see FIG. 1 B). This is consistent with the known capacity of this agent to preferentially boost Th1 responses. Cellular responses measured by ELISPOT, were also strongly enhanced by imiquimod applied topically to the immunization site (see FIG. 2A). No T-cell responses were detectable in mice immunized similarly to OVA alone. There was a slight response when imiquimod was applied 1×/wk following each immunization; but a very strong response when it was applied 3×/week.

This latter observation indicates the effect of imiquimod is dose and/or time dependent. It provides the foundation for one particular application of our invention which is to formulate imiquimod or other TLR ligand in a patch that can be applied to skin for several days. Incorporating the TLR ligand in a formulation that slowly releases the ligand from the patch might further improve the effectiveness of the patch. Similarly, in cases in which it is desired to administer a TLR ligand systemically (by intradermal, subcutaneous, or IM injection) with or without the vaccine it will be advantageous to incorporate the ligand in a slow released formulation (such as such as liposomes).

Additional experiments indicate topical imiquimod is a more potent stimulator of T-cell responses than IL-2 liposomes, the strongest adjuvant for CD8 responses we have studied to date (see FIG. 2B).

These results indicate that imiquimod is a potent vaccine adjuvant when applied topically. It can powerfully enhance both antibody and CD8 T-cells responses. The second major discovery that provides the foundation of this invention was a completely unexpected finding. It came to light in the course of control experiments conducted to confirm that imiquimod applied topically directly over the immunization site was an effective vaccine adjuvant.

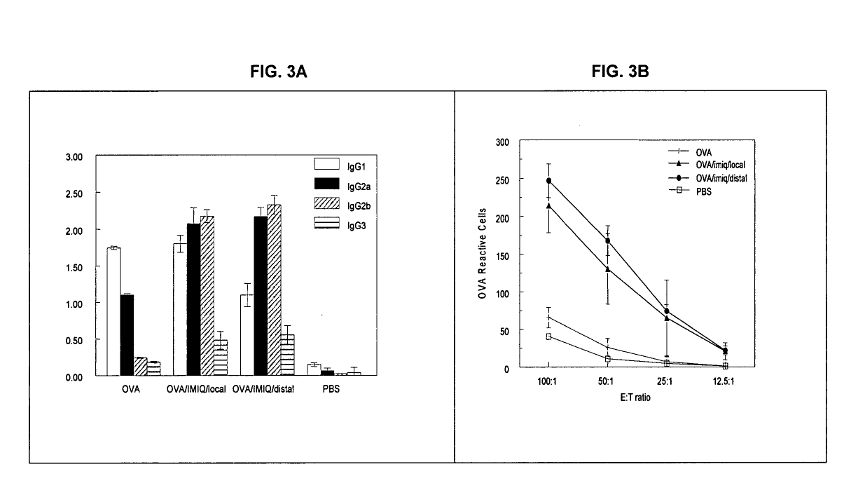

We found that imiquimod retained its strong vaccine adjuvant activity when applied topically to skin away from the site of vaccine immunization. As shown in FIG. 3, 5% imiquimod cream applied topically to skin over the nape of the neck markedly enhanced both humoral and cellular immune responses against an antigen injected sc in the lower abdominal region. In these experiments, groups of 0.6C57BI/6 mice were immunized sc weekly×4 to 0.1 mg of ovalbumin (OVA) given in 0.1 ml of PBS injected in the lower abdominal region. One group of mice was also treated with 5% imiquimod applied topically to the skin at a site distal (in the nape of the neck) to the immunization site 1×/week. (Control groups were immunized to PBS or to OVA. As shown in FIG. 3A, the anti-OVA IgG subclass response was considerably greater in mice immunized to OVA+topical imiquimod both local and distal to vaccine injection sites than in mice immunized to OVA alone. Endpoint titer analysis determined by ELISA pf serial serum dilutions showed that the IgG2a titers were 1:312,500 and 1:62,500 for local and distal imiquimod treatment and were 125and 25 times stronger than OVA alone respectively. The IgG2b endpoint titers were identical at 1:62,500 and were both 125 stronger than OVA alone.

Cellular responses measured by ELISPOT, were also strongly enhanced by imiquimod applied topically to both the local and distal immunization sites (see FIG. 3B). Both local and distal application of imiquimod produced strong cellular responses to OVA. By contrast, no significant T-cell responses above PBS controls were detectable in mice immunized similarly to OVA alone. We have not studied the adjuvant activity of other TLR receptor ligands such as resiquimod and CpG ODN applied topically away from the immunization site. However, all have been shown to enhance the immunogenicity of antigens when given systemically together with the vaccine and, as all are very small molecules, should penetrate through skin as well as imiquimod. Thus, it is reasonable to expect that all of these will also retain adjuvant activity when applied topically directly over, or away, from the immunization site. Because their specific mechanism of action differs from that of imiquimod, they may have stronger adjuvant activity or may be better able to enhance specific type of immune responses than imiquimod. Combinations of TLR ligands may be able to enhance stronger or broader type of immune response than any one individual TLR ligand alone. This invention is intended to cover these different possibilities.

Novel specific ways of using this invention include: 1) administering the TLR ligand topically away from the site of vaccine immunization as well as topically directly over the site of, but separately from, vaccine administration; 2) administering the TLR adjuvant orally or otherwise systemically (by injection or intranasaly) concurrently with vaccination; 3) enhancing the adjuvant activity of topical TLR ligand by applying them to skin in a patch or other slow release vehicle; 4) and administering the TLR ligand systemically in a slow release vehicle (such as liposomes). This invention applies not only to imiquimod, but to all other TLR7, 8 and 9 ligands.

The purpose of the invention is to provide novel methods of using a new class of potent adjuvants (toll-like receptor ligands) that: 1) provide the major advantage that the adjuvant can be used as a universal vaccine adjuvant that does not require the formulation of a new product with each new vaccine that the adjuvant is used with; and 2) permits the adjuvant to be used by topical administration over the site of vaccine immunization, or topically at a site distal from the vaccine or orally or by other systemic administration.

These novel approaches of using this class of adjuvants not only provides an adjuvant which appears to be more potent than ones currently in use, but can also result in major cost and time saving in the development and preparation of new vaccines, in stretching the supply of vaccines whose supply is limited, can simplify the administration of the vaccines, and increase their safety. It will also avoid the necessity to seek FDA approval of each new application of the adjuvant with a new vaccine. The adjuvant of the present inventor will also help to enhance the potency of human and animal vaccines, simplify the approval of new vaccine formulations, simplify and reduce the cost of vaccine production, and extend the supply of currently available vaccines.

The purpose of this invention is to provide novel methods of using a powerful new class of vaccine adjuvants (toll-like receptor [TLR] ligands). It provides the major advantage that the adjuvant can be used as a universal adjuvant that does not require the formulation of a new product with each new vaccine that the adjuvant is used with. This is accomplished by formulating the adjuvant as a separate product that is administered to the patient or animal independently, and at a separate site, from the vaccine. The separate site can be on the skin (topical application) or orally or systemically. The major advantages of this manner of using the adjuvants are that include:

1) The adjuvant is formulated and administered independently of the vaccine and at site different from that of vaccine administration. This is in contradicstinction to the conventional manner of using adjuvants, which is to admix them with the vaccine. The conventional approach means that each new formulation of the same adjuvant with a different vaccine is viewed as a new product which must go through the lengthy and expensive FDA certification process. In the present invention, the adjuvant is formulated and administered separately from the vaccine antigen, and hence is a separate product. As a result, the adjuvant can be used with a variety of vaccines without need to obtain FDA approval for each new use.
2) The formulation of the adjuvant can be optimized without concerns about the impact of the formulation on the vaccine antigens. The formulation of the vaccine itself can also be optimized without concern about the effects on the adjuvant.
3) The use of the adjuvant should be safer, as topical application of drugs is normally associated with fewer side effects than systemic administration.
4) It will reduce the cost of developing and producing vaccines, as it omits the cost of preparing, certifying, and validating formulations that contain both the antigen and the adjuvant. Only the antigen part of new vaccines would need to be validated and approved, not the adjuvant part and/or the combination.
5) TLR ligand adjuvants appear more powerful than many current adjuvants. They can enhance the effectiveness of current vaccines and/or permit lesser amounts to be used to obtain the same effect. This latter property would be very helpful to extend the availability of vaccines that are in short supply, such as the current influenza vaccines.
6) There are a variety of TLR ligands. These include ligands which can react with a variety of TLRs (TLR 7, 8 and 9 for example) and different ligands that react against each TLR. Each of these ligands may have somewhat different effects on the stimulation of immune responses by the vaccine. Based on the TLR stimulated and on the ligand, the antibody and T-cell responses and various parameters of each type of these responses may be enhanced to different degree.

It should be apparent to a person of ordinary skill in the art upon review of the foregoing detailed description of the preferred embodiments that many modifications and improvements may be made to the present invention without departing from the sprint or scope thereof. It is therefore intended that the invention be defined by the following claims.

### REFERENCES

The following are incorporated herein by reference:
1. Immier, J. L., Zheng, L., “Biology of Toll Receptors: Lessons from Insects and Mammals.” J. Leukoc. Biol. 75:18-26, 2004.
2. Hengge, U. R., Ruzicka, T., “Topical Immunomodulation in Dermatology: Potential of Toll-like Receptor Agonists.” Dermatol Surg. 30:1101-1112, 2004.
3. Klinman, D. M., “Immunotherapeutic Uses of CpG Oligodeoxybuckeotides.” Nature Reviews 4:1-8, 2004.
4. Horning, V., S. Rothenfusser, S. Britsch, A. et al., “Quantitative Expression of Toll-like Receptor 1-10 mRNA in Cellular Subsets of Human Peripheral Blood Mononuclear Cells and Sensitivity to CpG Oligodeoxynucleotides.” J. Immunol. 168:4531, 2002.
5. Ito, T., R. Amakawa, T. Kaisho, H. et al., “Interferon-a and Interleukin-12 are Induced Differentially by Toll-like Receptor 7 Ligands in Human Blood Dendritic Cell Subsets.” J. Exp. Med. 195:1507, 2002.
6. Lore, K., Beffs, M. R., Brenchley, J. M., et al., “Toll-Like Receptor Ligands Modulate Dendritic Cells to Augment Cytomegalovirus-and HIV-1-Specifice T Cell Responces.” J. Immunol. 171:4320, 2003.
7. Krug, A., A. Towarowski, S. Britsch, S. et al., “Toll-like Receptor Expression Reveals CpG DNA as a Unique Microbial Stimulus for Plasmacytoid Dendritic Cells which Synergizes with CD40 Ligand to Induce High Amounts of IL-12.” Eur. J. Immunol. 31:3026, 2001.
8. Hemmi, H., T. Kaisho, O. Takeuchi, S. Sato, et al., “Small Anti-viral Compounds Activate Immune Cells via the TLR7 MyD88-Dependent Signaling Pathway.” Nat. Immunol. 3:196, 2002.
9. Jurk M., F. Heil, J. Vollmer, C., et al., “Human TRL7 or TRL8 Independently Confer Responsiveness to the Antiviral Compound R-848.” Nat. Immonol. 3:499, 2002.
10. Bauer, M., V. Redecke, J. W. Ellwart, et al., “Bacterial CpG-DNA Triggers Activation and Maturation of Human CD11c-CD123+ Dendritic Cells.” J. Immonol. 166:5000, 2001.
11. Ito, T., R. Amakawa, T. Kaisho, et al., “Interferon-a and Interleukin-12 are Induced Differentially by Toll-like Receptor 7 Ligands in Human Blood Dendritic Cell Subsets.” J. Exp. Med. 195:1507, 2002.
12. Gibson, S. J., Lindh, J. M., Riter, T. R., et al., “Plasmacytoid Dendritic Cells Produce Cytokines and Mature in Response to the TLR 7 Agonists Imiquimod and Resiquimod.” Cellular Immun. 318:74-86, 2002.
13. Thomsen, L., L., Topley, P., Daly, M. G., et al., “Imiquimod and Resiquimod in a Mouse Model Adjuvants for DNA Vaccination by Particle-medicated Immunotherapeutic Delivery.” Science Direct Vaccine 22:1799-1809, 2004.
14. Palamara, F., Meindl, S., Holmann, M., et alilia, M., “Identification and Characterization of pDC-like Cells in Normal Mouse Skin and Melanomas Treated with Imiquimod.” J. Immunol. 173:3051-306, 2004.
15. Imbertson, L. M., Beaurline, J. M., Couture, A. M., et. al., “Cytokine Induction in Hairless Mouse and Rat Skin after Topical Application of the Immune Response Modifiers Imiquimod and S-28463.” J. Invest. Dermatol. 110:734, 1998.
16. Klinman, D. M., “Immunotherapeutic Uses of CpG Oligodeoxybuckeotides.” Nature Reviews Volume 4:1-8, 2004.
17. Klinman, D. M., Barnhart, K. M., Cronover, J., “CpG Motifs as Immune Adjuvants.” Vaccine 17:19-25, 1999.
18. Brazalot, M, Milian, C. L., Weeratna, R., et al., “CpG DNA can induce strong T_{H}1 Humoral and Cell-Mediated Immune Responses Against Hepatitis B Surface Antigen in Young Mice.” Proc. Natl. Acad. Sci. USA, 95:15553-15558, 1998.
19. Eastcott, J. W., et. al., “Oligonucleotide Containing CpG Motifs Enhances Immune Response to Mucosally or Systematically Administered Tetanus Toxoid.” Vaccine 19:1636-1642 2001.
20. Davis, H. L. et al., “CpG DNA Overcomes Hyporesponsiveness to Hepatitis B Vaccine in Orangutans.” Vaccine 18:1920-1924, 2000.
21. Jones, T. R., et. al., “Synthetic Oligodeoxynucleotides as Adjuvants.” Vaccine 19:413-422 1999.
22. Verthelyi, D., et. al., “CpG Oligodeoxynucleotides as Vaccine Adjuvants in Primates.” J. Immunol. 168:1659-1663, 2002.
23. Ulhmann, E., Vollmer, J., “Recent Advances in the Development of Immunostimulatory Oligonucleotides.” Curr Opin. Drug Disc. Devel. 6:204-17, 2003.
24. Bernstein, D. I., Miller, R. L., Harrison, C. J., “Adjuvant Effect of Immiquimod on a Herpes Simplex Virus Type 2 Glycoprotein Vaccine in Guinea Pigs.” J. Infect Dis. Mar; 167: 731-5, 1993.
25. Otero, M., Calarota, S. A., Felber, F., Laddy, D., Pavlakis, G., Boyer, J. D., Weiner, D. B., “Resiquimod is a Modest Adjuvant for HIV-1 Gag-based Genetic Immunization in a Mouse Model,” Science Direct Vaccine Volume 22:1782-1790, 2004.
26. Kadowaki, N., S. Ho, S. Antonenko, R. W. Malefyt, R. A. Kastelein, F. Bazan, Y. J. Liu., “Subsets of Human Dendritic Cell Precursors Express Different Toll-like Receptors and Respond to Different Microbial Antigens,” J. Exp. Med 194:863, 2001.
27. Jarrossay, D., G. Napolitani, M. Colonna, F. Sallusto, A. Lanzavecchia, “Specialization and Complimentarity in Microbial Molecule Recognition by Human Myeloid and Plasmacytoid Dendritic Cells,” Eur. J. Immunol. 31:3388, 2001.
28. Bystryn J-C. “Antibody Response and Tumor Growth in Syngeneic Mice Immunized to Partially Purified B16 Melanoma Associated Antigens,” J. Immunol 120:96-101, 1978.

I claim:

1. A method for increasing immunological response to a vaccine, comprising:
administering the vaccine subcutaneously to a patient in need thereof; and
administering a topical composition containing an amount of a toll like receptor ligand effective to increase immune response of the patient to the vaccine.

2. The method of claim 1, wherein the toll like receptor ligand is imiquimod or resiquimod.

3. The method of claim 1, wherein the topical composition includes imiquimod or resiquimod, and wherein the topical composition is applied to the skin of the patient after administering the vaccine to the patient.

4. The method of claim 1, wherein the topical composition contains about 1 percent to 10 percent by weight imiquimod or resiquimod in a pharmaceutically effective vehicle, and the topical composition is applied to a site on the patient remotely from where the vaccine is administered.

5. The method of claim 1, wherein the topical composition contains about 1 percent to about 5 percent by weight imiquimod in a pharmaceutically acceptable vehicle.

6. The method of claim 5, wherein the topical composition is applied to a site distal from where the vaccine was administered.

7. The method of claim 5, wherein the topical composition is applied to a site where the vaccine was administered.
